(19) United States
(12) Patent Application Publication
(10) Pub. No.: US 2025/0281060 A1
(43) Pub. Date: Sep. 11, 2025

(54) SYSTEMS AND METHODS FOR DETERMINING A FLOW SPEED OF A FLUID FLOWING THROUGH A CARDIAC ASSIST DEVICE

(71) Applicant: Kardion GmbH, Stuttgart (DE)

(72) Inventors: Thomas Alexander Schlebusch, Renningen (DE); Tobias Schmid, Stuttgart (DE)

(21) Appl. No.: 19/218,061

(22) Filed: May 23, 2025

Related U.S. Application Data
(63) Continuation of application No. 15/734,523, filed on Oct. 15, 2021, now Pat. No. 12,310,708.

(30) Foreign Application Priority Data
Jun. 6, 2018 (DE) 102018208933.7

(51) Int. Cl. A61B 5/029; A61B 5/00; A61B 8/00; A61B 8/06; A61M 60/178; A61M 60/226; A61M 60/237; A61M 60/523

(52) U.S. Cl. CPC A61B 5/029; A61B 5/686; A61B 5/6869; A61B 8/065; A61B 8/488; A61M 60/178; A61M 60/226; A61M 60/237; A61M 60/523; A61M 2205/3334; A61M 2205/3375

(57) ABSTRACT

The invention relates to a method for determining at least a flow velocity or a fluid volume flow of a fluid flowing through an implanted vascular support system, comprising the following steps: a) carrying out a pulsed Doppler measurement by means of an ultrasonic sensor of the support system, b) evaluating a measurement result from step a), which has a possible ambiguity, c) providing at least one operating parameter of a flow machine of the support system, d) determining at least the flow velocity or the fluid volume flow using the measurement result evaluated in step b), wherein the possible ambiguity of the measurement result is corrected using the operating parameter.

### INCORPORATION BY REFERENCE TO ANY PRIORITY APPLICATIONS

Any and all applications for which a foreign or domestic priority claim is identified in the Application Data Sheet as filed with the present application are hereby incorporated by reference under 37 CFR 1.57. This application is a continuation of U.S. patent application Ser. No. 15/734,523, entitled SYSTEMS AND METHODS FOR DETERMINING A FLOW SPEED OF A FLUID FLOWING THROUGH A CARDIAC ASSIST DEVICE, and filed Oct. 15, 2021, which is the U.S. National Phase of international application number PCT/EP2019/064807, entitled METHOD FOR DETERMINING A FLOW SPEED OF A FLUID FLOWING THROUGH AN IMPLANTED, VASCULAR ASSISTANCE SYSTEM AND IMPLANTABLE, VASCULAR ASSISTANCE SYSTEM, and filed Jun. 6, 2019, which is an International Application of and claims the benefit of priority to German Application No. 102018208933.7, filed Jun. 6, 2018, the disclosure of each of which is hereby incorporated by reference herein in its entirety for all purposes and forms a part of this specification.

### BACKGROUND

### Field

The invention relates to a method for determining a flow velocity of a fluid flowing through an implanted vascular support system, an implantable vascular support system, and a use of an operating parameter of a flow machine of an implanted vascular support system. The invention is in particular used in (fully) implanted left-heart support systems (LVAD).

### Description of the Related Art

It is known to integrate ultrasonic volume flow sensors into heart support systems in order to therewith detect the so-called pump volume flow, which quantifies the fluid volume flow through the support system itself. The ultrasonic volume flow sensors can carry out pulsed Doppler measurements or use the pulsed Doppler (pulsed wave Doppler; in short: PWD) method. This method requires only one ultrasound transducer element and allows precise selection of the distance of the observation window from the ultrasound element.

The task of a cardiac support system is to convey blood. In this case, the so-called heart-time volume (HTV, usually indicated in liters per minute) is highly clinically relevant. In other words, the heart-time volume in this case relates to the total volume flow of blood from a ventricle, in particular from the left ventricle, to the aorta. Correspondingly clear is the attempt to collect this parameter as a measured value during operation of a cardiac support system.

Depending on the level of support, which describes the proportion of volume flow conveyed by a conveying means, such as a pump of the support system, to the total volume flow of blood from the ventricle to the aorta, a certain volume flow reaches the aorta via the physiological path through the aortic valve. The heart-time volume or the total volume flow (Q_{HTV}) from the ventricle to the aorta is therefore usually the sum of the pump volume flow (Qₚ) and the aortic valve volume flow (Qₐ).

An established method for the determination of the heart-time volume (Q_{HTV}) in the clinical setting is the use of dilution methods, which, however, all rely on a catheter inserted transcutaneously and therefore can only provide heart-time volume measurement data during cardiac surgery and during the subsequent stay in intensive care. For high levels of support, Qₐ approaches zero so that approximately Qₚ≠Q_{HZV} applies. Accordingly, at least in these cases, the heart-time volume can be determined at least approximately via the pump volume flow. An established method for measuring the pump volume flow (Qₚ) is the correlation of the operating parameters of the support system, predominantly the electrical power consumption, possibly supplemented by further physiological parameters, such as the blood pressure. Since these methods are based on statistical assumptions and the underlying pump characteristic map of the support system used, the correlated Qₚ are error-prone. In order to increase the measurement quality of the parameter Qₚ, the inclusion of a flow sensor is therefore desirable.

A particularly suitable sensor method for determining flow velocities and thus also volume flows is ultrasound, in particular the pulsed wave Doppler method (PWD) since it requires only one bidirectional ultrasound transducer element and allows precise selection of the distance of the observation window in which the measured values are collected. It is thus possible to carry out the flow velocity measurement in the range in which suitable flow conditions prevail.

In a PWD system, ultrasonic pulses are sent out at a defined pulse repetition rate (PRF). If the flow velocity and flow direction are unknown, the PRF must exceed at least twice the maximum occurring Doppler frequency shift in order to not violate the Nyquist theorem. If this condition is not met, aliasing occurs, i.e., ambiguities in the determined frequency spectrum. When detecting a frequency in the frequency spectrum, it can no longer be unambiguously assigned to one but several flow velocities.

Due to the geometric design of the measurement setup in heart support systems (VAD), the measurement range or the observation window may be so far away from the ultrasound transducer that the signal transit time of the ultrasonic pulse from the transducer to the measurement range and back to the transducer is not negligible. Since a new ultrasonic pulse may only be sent out if the preceding one no longer delivers significant echoes, the signal transit time limits the maximum possible PRF. In the case of the high flow velocities prevailing in heart support systems and the geometric boundary conditions for the distance of the observation window from the ultrasound element, there is inevitably a violation of the Nyquist sampling theorem, which results in ambiguities being produced in the spectrum.

Heart support systems with ultrasonic sensors that do not use the PWD method are usually equipped with two ultrasound transducers so that the described transit time problem can occur but can be solved otherwise with appropriate implementation. However, heart support systems with ultrasonic sensors that use the PWD method are susceptible to the described effect, in particular in the case of moderate to high flow velocities. The current state of the art is the measure to select the defined pulse repetition rate such that aliasing does not occur or to suitably adjust, if possible, both the geometric conditions and the ultrasound frequency.

### SUMMARY

The object of the invention is to specify an improved method for determining a flow velocity of a fluid flowing through an implanted vascular support system and to create an improved implantable vascular support system in which the flow velocity of a fluid flowing through it can be determined.

In particular, it is an object of the invention to create a method for determining a flow velocity of a fluid and an improved implantable vascular support system, in which the determination of the flow velocity of a fluid flowing through it is provided, in which the determination of the flow velocity at the flow velocities prevailing in a heart support system is possible with only one ultrasound transducer, even in the case of a long signal transit time of an ultrasonic pulse from the ultrasound transducer to the measurement range and back.

This object is achieved by the systems and methods disclosed herein.

Advantageous embodiments of the invention are specified herein.

In some embodiments, a method for determining at least a flow velocity or a fluid volume flow of a fluid flowing through an implanted vascular support system is proposed here, comprising the following steps:
a) carrying out a pulsed Doppler measurement by means of an ultrasonic sensor of the support system,
(b) evaluating a measurement result from step a), which has a possible ambiguity,
(c) providing at least one operating parameter of a flow machine of the support system,
(d) determining at least the flow velocity or the fluid volume flow using the measurement result evaluated in step b),
wherein the possible ambiguity of the measurement result is corrected using the operating parameter.

The vascular support system is preferably a cardiac support system, particularly preferably a ventricular support system. The support system is regularly used to support the conveyance of blood in the blood circulation of a person, if appropriate patients. The support system can be arranged at least partially in a blood vessel. The blood vessel is, for example, the aorta, in particular in the case of a left-heart support system, or the common trunk (Truncus pulmonalis) into the two pulmonary arteries, in particular in the case of a right-heart support system. The support system is preferably arranged at the exit of the left ventricle of the heart or the left heart chamber. The support system is particularly preferably arranged in the aortic valve position.

The solution proposed here contributes in particular to providing an aliasing compensation method for an ultrasonic volume flow sensor in a heart support system. The method can contribute to determining a fluid flow velocity and/or a fluid volume flow from a ventricle of a heart, in particular from a (left) ventricle of a heart, to the aorta in the region of a (fully) implanted (left) ventricular (heart) support system. The fluid is regularly blood. The flow velocity is determined in a fluid flow or fluid volume flow, which flows through the support system, in particular through an (inlet) tube or an (inlet) cannula of the support system. The method advantageously allows the flow velocity and/or the fluid volume flow of the blood flow to be determined with high quality even outside the surgical scenario, in particular by the implanted support system itself.

The solution proposed here can particularly advantageously use the fact that based on the motor characteristic map, a rough estimation of the pump flow is possible (only) from the rotation rate of the drive or on the basis of the differential pressure across the flow machine and the rotation rate. The in particular rough estimate of the flow rate from the operating parameters of the flow machine is used in particular to resolve the ambiguities in the spectrum and to enable highly precise flow measurement by the ultrasonic sensor.

In step a), a pulsed Doppler measurement is carried out by means of an ultrasonic sensor of the support system. In order to carry out the pulsed Doppler measurement, the pulsed Doppler (pulsed wave Doppler; in short: PWD) method is in particular used. In particular, a PWD measurement cycle is run through in step a).

In step b), a measurement result from step a) which has a possible ambiguity is evaluated. “Possible ambiguity” means in other words in particular that the measurement result or all measurement results do not necessarily always have to have an ambiguity. In particular, in the case of a comparatively high flow velocity, as commonly occurring in the support systems in question here, the measurement result generally has an ambiguity. However, at a comparatively low flow velocity, it can also happen that the measurement result is unambiguous.

The measurement result can furthermore be provided in particular after step b). In this case, the measurement result can, for example, be provided as raw data (e.g., frequency spectrum) or as raw measurement result or as already at least partially preprocessed measurement result (e.g., as a (measured) flow velocity and/or as a (measured) fluid volume flow). The measurement result can be provided to a processing unit of the support system, for example.

In step c), at least one operating parameter of a flow machine of the support system is provided. The operating parameter can be provided to a processing unit of the support system, for example. The measurement result provided in step b) and the operating parameter provided in step c) are generally detected with respect to the same fluid flow, e.g., in the same (temporal and/or spatial) observation window. In other words, this means in particular that the measurement result provided in step b) and the operating parameter provided in step c) relate to substantially the same measurement time or have substantially the same time stamp and/or relate to the same measuring point. In this case, “substantially” in particular describes a deviation of less than one second. A time difference (generally of less than one second) can be taken into account until the operating parameter (or a change thereof) affects the measuring point. This can also be described in such a way that the measurement result provided in step b) and the operating parameter provided in step c) are associated with each other. At least one operating parameter associated with the measurement result provided in step b) is preferably provided in step c).

In step d), the (actual) flow velocity is determined using the measurement result evaluated in step b). If a raw measurement result is evaluated in step b) and then provided, it is particularly advantageous if a (measured) flow velocity is determined therefrom (e.g., in step d). If a pre-processed measurement result for a (measured) flow velocity is provided in step b), it can advantageously be used directly in step d). The (measured) flow velocity is generally not unambiguous. Furthermore, it is advantageous if an estimated flow velocity is determined on the basis of the operating parameter provided in step c). The (actual) flow velocity can now be determined, for example, by selecting the measured flow velocity that is closest to the estimated flow velocity.

Alternatively or cumulatively, an (actual) fluid volume flow (instead of the flow velocity) can be determined in step d). If a raw measurement result is provided in step b), it is particularly advantageous if a (measured) fluid volume flow is determined therefrom. If a pre-processed measurement result for a (measured) fluid volume flow is provided in step b), it can advantageously be used directly in step d). The (measured) fluid volume flow is generally not unambiguous. Furthermore, it is advantageous if an estimated fluid volume flow is determined on the basis of the operating parameter provided in step c). The (actual) flow volume flow can now be determined, for example, by selecting the measured fluid volume flow that is closest to the estimated fluid volume flow.

In the sense of the solution proposed here, the possible ambiguity of the measurement result is corrected or resolved using the operating parameter. The measurement result is generally ambiguous. This ambiguity can be explained in particular by the generally present violation of the Nyquist sampling theorem in this case. This violation of the Nyquist sampling theorem is caused in particular by comparatively long signal transit times existing in the support system between the ultrasonic sensor and the observation window or measurement range and a new ultrasonic pulse in the pulsed Doppler measurements generally being sent out only if the echo of an ultrasonic pulse sent out immediately beforehand was received or has died away.

The possible ambiguity can, for example, be corrected or resolved in step d). In this context, the flow velocity can be determined in step d) using the (possibly ambiguous) measurement result evaluated and/or provided in step b) and the operating parameter provided in step c), wherein the possible ambiguity of the measurement result is corrected using the operating parameter. A possibility to carry out such a correction or to resolve a possible ambiguity has already been described above. By way of example, the measured flow velocity or the measured fluid volume flow that is closest to the estimated flow velocity or the estimated fluid volume flow is selected in this case.

Alternatively, the possible ambiguity can (already) be corrected or resolved in step b), for example. This alternative can also be referred to as a priori estimation or as a priori selection or pre-selection. In other words, this means in particular that the possible ambiguity is already corrected or resolved during the evaluation of the measurement result. This can be particularly advantageous take place such that (only) the range or section of the (raw) measurement result in which a plausible result is to be expected is evaluated. The evaluated (no longer ambiguous) measurement result can in this case be provided in step b). The evaluated (no longer ambiguous) measurement result can in this case be used in step d).

“A priori” here means in particular that the operating parameter is provided and/or the estimated flow velocity or the estimated fluid volume flow is determined before the (possibly ambiguous) measurement result is evaluated (and, if applicable, provided). For example, the operating parameter, the a priori estimated flow velocity and/or the a priori estimated fluid volume flow (possibly in the form of a window function or windowing) can contribute to a pre-selection in order to evaluate and/or provide only a plausible measurement result or only the plausible part of the measurement result. For this purpose, a (reflected and then) received ultrasonic pulse can, for example, only be evaluated in the (frequency) section in which a plausible result is to be expected.

According to an advantageous embodiment, it is proposed that a new ultrasonic pulse is only sent out in step a) if an echo of an ultrasonic pulse sent out immediately beforehand has (sufficiently) died away and/or was received. A new ultrasonic pulse is preferably sent out only if all (significant) echoes of an ultrasonic pulse sent out immediately beforehand have (sufficiently) died away and/or were received. A new ultrasonic pulse is furthermore preferably sent out only if the (significant) echoes of an ultrasonic pulse sent out immediately beforehand from a (predefined) measurement window or measurement range have (sufficiently) died away and/or were received.

According to an advantageous embodiment, it is proposed that a maximum pulse repetition rate of the pulsed Doppler measurement is less than two times a maximum occurring Doppler shift. The maximum pulse repetition rate of the pulsed Doppler measurements is preferably less than the maximum occurring or expected Doppler shift. If the maximum pulse repetition rate is less than twice the maximum occurring Doppler shift, the Nyquist sampling theorem is in principle violated. However, this violation may be necessary to perform a PWD method in a vascular support system.

According to an advantageous embodiment, it is proposed that the operating parameter is at least one rotational speed, one current, one power, or one pressure. The operating parameter is preferably a rotational speed (or rotation rate) of the flow machine, e.g., of a drive (e.g., of an electric motor) and/or of a paddle wheel of the flow machine. The at least one operating parameter furthermore preferably comprises a rotational speed of the flow machine and a differential pressure across the flow machine.

According to an advantageous embodiment, it is proposed that a plausible range in which plausible measurement results can be located is (a priori) determined using the operating parameter. In this context, a window function or windowing can be used in the frequency analysis (e.g., by means of discrete Fourier transformation) of the (reflected and then) received ultrasonic pulse. A so-called Hamming window is preferably used. The windowing, in particular the Hamming window, can advantageously be formed as a function of the operating parameter and/or the expected and/or estimated flow velocity (on the basis of the operating parameter) and/or the expected and/or estimated fluid volume flow (on the basis of the operating parameter).

According to an advantageous embodiment, it is proposed that a fluid volume flow through the support system is determined using the flow velocity. In other words, this relates in particular to a fluid volume flow which flows (only) through the support system itself, e.g., through an (inlet) tube or an (inlet) cannula of the support system. The fluid volume flow is usually the so-called pump volume flow (Qₚ), which only quantifies the flow through the support system itself. If this value is known in addition to the total volume flow or heart-time volume (Q_{HZV}), the so-called level of support can be calculated from the ratio of Qₚ to Q_{HZV} (i.e., Qₚ/Q_{HZV}). In order to determine the fluid volume flow, the determined flow velocity can be multiplied, for example, with a flow cross section of the support system, in particular a tube or cannula flow cross section.

According to a further aspect, an implantable, vascular support system is, comprising:
an ultrasonic sensor configured to carry out a pulsed Doppler measurement,
a flow machine,
a processing unit configured to correct a possible ambiguity of a measurement result of the ultrasonic sensor using the operating parameter of the flow machine.

The support system is preferably a left ventricular heart support system (LVAD) or a percutaneous, minimally invasive left-heart support system. Furthermore, the support system is preferably fully implantable. In other words, this means in particular that the means required for the detection, in particular the ultrasonic sensor, are completely located in the body of the patient and remain there. The support system can also be designed in multiple parts or with several components that can be arranged at a distance from one another, so that, for example, the ultrasonic sensor and the processing unit (measuring unit) can be separated from one another by a cable. In the multi-part design, the processing unit arranged separately from the ultrasonic sensor can also be implanted or arranged outside the body of the patient. In any case, it is not absolutely necessary for the processing unit to also be arranged in the body of the patient. For example, the support system can be implanted such that the processing unit is arranged on the skin of the patient or outside the body of the patient and a connection is established to the ultrasonic sensor arranged in the body. The support system is particularly preferably configured and/or suitable for being arranged at least partially in a ventricle, preferably in the left ventricle of a heart, and/or in an aorta, in particular in the aortic valve position.

The support system furthermore preferably comprises a tube (or a cannula), in particular an inlet tube or inlet cannula, a flow machine, such as a pump and/or an electric motor. The electric motor is regularly a component of the flow machine. The (inlet) tube or the (inlet) cannula is preferably configured such that in the implanted state, it can guide fluid from a (left) ventricle of a heart to the flow machine. The support system is preferably elongated and/or tubular. The tube (or the cannula) and the flow machine are preferably arranged in the region of opposite ends of the support system.

In particular, precisely or only one ultrasonic sensor is provided. The ultrasonic sensor preferably comprises precisely or only one ultrasound transducer element. This is in particular sufficient for a Doppler measurement if the PWD method is used.

The flow machine is preferably designed at least in the manner of a pump or an (axial or radial) compressor. The flow machine can provide at least one of its (current) operating parameters of the processing unit. In addition, a control unit for controlling or regulating the flow machine can be provided, which, for example, controls or regulates at least one rotational speed or one power of the flow machine as a function of (among other things) a flow velocity determined by way of example by the processing unit.

The support system is preferably configured to carry out a method proposed here.

According to a further aspect, a use of an operating parameter of a flow machine of an implanted vascular support system for correcting a possible ambiguity of a measurement result of an ultrasonic sensor of the support system is proposed. Preferably, at least one method proposed here or a support system proposed here is used for correcting a possible ambiguity of a measurement result of an ultrasonic sensor.

The details, features, and advantageous embodiments discussed in connection with the method can also arise accordingly in the support system presented here and/or in the use and vice versa. In this respect, reference is made in full to the explanations there regarding the detailed characterization of the features.

### BRIEF DESCRIPTION OF THE DRAWINGS

The solution presented here as well as its technical environment are explained in more detail below with reference to the figures. It should be pointed out that the invention is not to be limited by the exemplary embodiments shown. In particular, unless explicitly stated otherwise, it is also possible to extract partial aspects of the facts explained in the figures and to combine them with other components and/or insights from other figures and/or the present description. The following are shown schematically:

FIG. 1 an implantable vascular support system,

FIG. 2 the support system according to FIG. 1 implanted in a heart,

FIG. 3 a further implantable vascular support system,

FIG. 4 the support system according to FIG. 3 implanted in a heart,

FIG. 5 an exemplary illustration of a Doppler measurement,

FIG. 6 a sequence of a method presented here in a normal operating procedure,

FIG. 7 an exemplary Doppler frequency spectrum,

FIG. 8 a further exemplary Doppler frequency spectrum, and

FIG. 9 a functional illustration of a possible embodiment of the method presented here.

### DETAILED DESCRIPTION

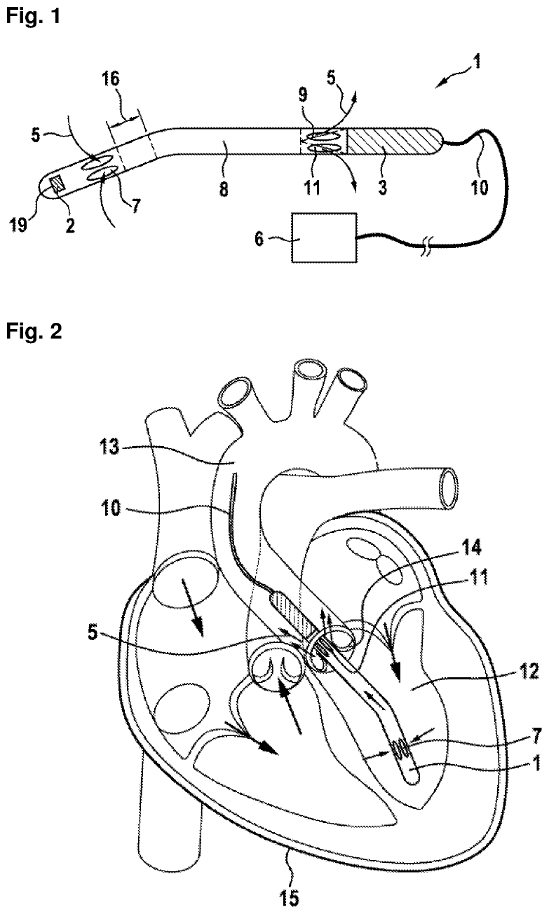

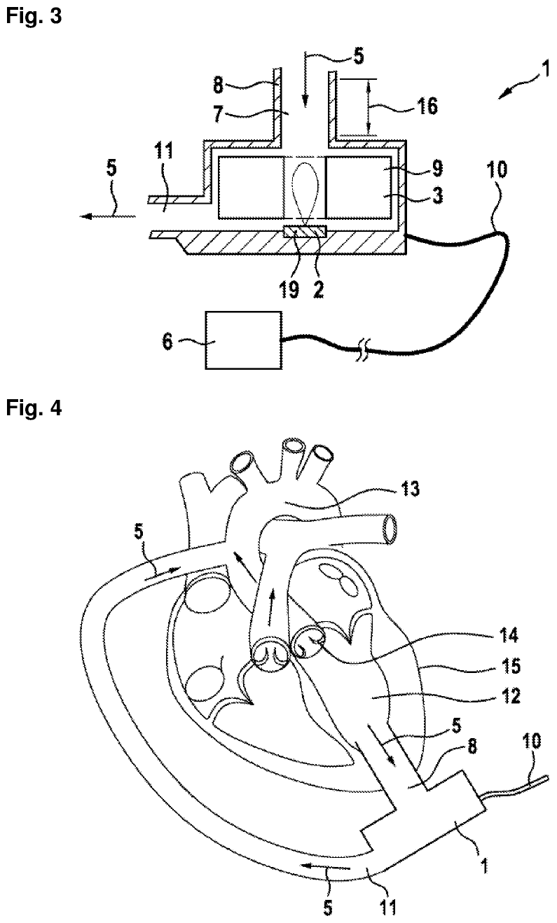

The vascular support system is preferably a ventricular and/or cardiac support system or a heart support system. Two particularly advantageous forms of heart support systems are systems positioned in the aorta according to FIG. 2 and systems positioned apically according to FIG. 4. The respective systems are explained in more detail in connection with FIG. 1 (aortic) and FIG. 3 (apical).

FIG. 1 schematically shows an implantable vascular support system 1. FIG. 1 illustrates an embodiment of an aortically positioned (cf. FIG. 2) or positionable support system 1. The support system 1 comprises an ultrasonic sensor 2 configured to carry out a pulsed Doppler measurement, a flow machine 3, and a processing unit 6 configured to correct a possible ambiguity of a measurement result of the ultrasonic sensor 2 using the operating parameter of the flow machine 3. The ultrasonic sensor 2 in this case comprises by way of example precisely one ultrasound (transducer) element 19.

In this case, the support system 1 according to FIG. 1 furthermore comprises, by way of example, a distal part with inlet openings 7 through which the blood can be drawn into the interior of the system, and an inlet tube 8 (which is formed in the manner of an inlet cannula in the aortic embodiment according to FIG. 1). In addition, the flow machine 3 is equipped by way of example with an impeller 9. In this case, a supply cable 10 is positioned by way of example proximally to the drive (e.g., electric motor; not shown here) of the flow machine 3. In the region of the impeller 9, there are also outlet openings 11, through which the blood can be discharged. During operation, a fluid volume flow 5 flows through the inlet tube 8, said fluid volume flow entering the support system 1 via the inlet openings 7 and exiting again via the outlet openings 11. This fluid volume flow 5 can also be referred to as a so-called pump volume flow.

FIG. 2 schematically shows the support system 1 according to FIG. 1 implanted in a heart 15. The reference signs are used uniformly so that reference can be made to the above explanations.

The inlet openings 7 are located in the implanted state, for example, in the region of the ventricle 12, while the outlet openings are located in the implanted state in the region of the aorta 13. This orientation of the support system 1 is merely exemplary here and not mandatory; rather, the support system can be oriented in the reverse direction, for example. In this case, the system is furthermore implanted by way of example in such a way that it passes through the aortic valve 14. Such an arrangement can also be referred to as a so-called aortic valve position.

FIG. 3 schematically shows a further implantable vascular support system 1. FIG. 3 illustrates an embodiment of an apically positioned (cf. FIG. 4) or positionable support system 1. The functioning of an apically implanted system is in principle comparable so that uniform reference signs can be used for all components in this case. Reference is therefore made here to the above explanations regarding FIG. 1.

FIG. 4 schematically shows the support system 1 according to FIG. 3 implanted in a heart 15. The reference signs are used uniformly so that reference can also be made here to the above explanations.

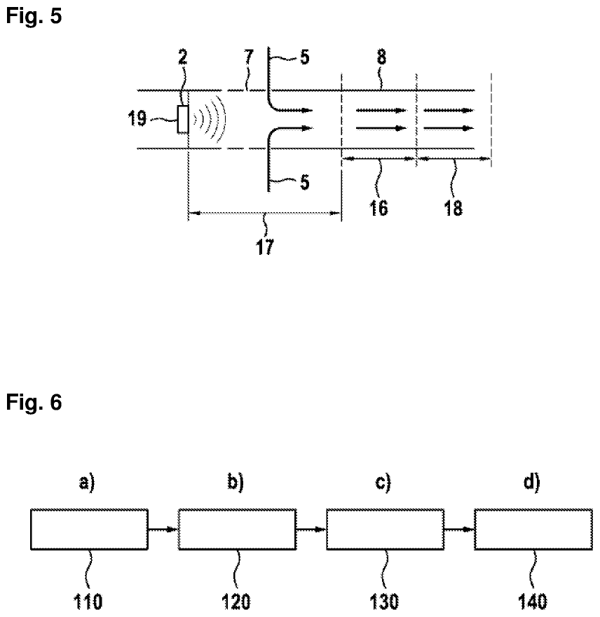

FIG. 5 schematically shows an exemplary illustration of a Doppler measurement. For this purpose, the ultrasonic sensor 2 of the support system 1 according to FIG. 1 is used by way of example in order to carry out a measurement in an inlet tube 8 of the support system 1 according to FIG. 1.

The measurement window, also referred to as the observation window and/or measurement range, for the ultrasound measurement is marked in FIGS. 1, 3, and 5 with reference sign 16. The selection of the measurement window 16 depends on the specific design of the (heart) support system 1 and should in principle be positioned where suitable flow conditions prevail. For example, FIG. 5 shows a simplified sectional view of the distal end of the embodiment of FIG. 1. In this case, it is shown schematically that no parallel flow lines prevail to the left of the measurement window 16 in the range 17. Since the Doppler effect is also a function of the cos (a) between the main beam direction of the ultrasound transducer and the main flow direction, it is advantageous to measure in a range of parallel flow lines. Although a measurement window (e.g., range 18) positioned too far away is possible in principle, it can intensify the aliasing effect explained below and/or provide strong attenuation of the ultrasound signal.

The ultrasonic sensor 2 is configured to carry out a pulsed Doppler measurement. The pulsed Doppler (pulsed wave Doppler; in short: PWD) method is basically used for ultrasound measurement in this case. The ultrasonic sensor 2 and the processing unit 6 can therefore also be referred to below as a so-called PWD system.

The measurement window 16 can typically be selected electronically in the PWD system so that a statement about the flow conditions in different regions of the flow guidance can also advantageously be made by means of measurement windows 16 of different depths.

In the (apical) embodiment according to FIG. 4, the blood flows toward the ultrasound element 19 in the opposite direction. The rotating impeller 9 is located between ultrasound element 19 and inlet tube 8. In this case, strong turbulence in the blood flow is to be expected so that it is also particularly advantageous here to position the measurement window 16 in front of the impeller 9, approximately in the region of the inlet tube 8.

The relatively high flow velocities in the range of the measurement window 16 in relation to the distance of the ultrasound (transducer) element 19 from the measurement window 16 have a great influence on the PWD application in both (heart) support system variants, predominantly in the (aortic) variant according to FIG. 1.

FIG. 6 schematically shows a sequence of a method presented here in a normal operating procedure. The method is used to determine at least a flow velocity or a fluid volume flow of a fluid flowing through an implanted vascular support system 1 (cf. FIGS. 1 to 5). The illustrated order of the method steps a), b), c), and d) with the blocks 110, 120, 130, and 140 is only exemplary. In block 110, a pulsed Doppler measurement is carried out by means of an ultrasonic sensor 2 of the support system 1. In block 120, a measurement result from step a) which has a possible ambiguity is evaluated. In block 130, at least one operating parameter of a flow machine 3 of the support system 1 is provided. In block 140, at least the flow velocity or the fluid volume flow is determined using the measurement result evaluated in step b). In the method, the possible ambiguity of the measurement result is corrected using the operating parameter.

For an exemplary illustration of the method, a system according to FIG. 1 with the following parameters is assumed:
inner diameter of the inlet tube: 5 mm
maximum blood flow to be measured: 9 liters/minute
resulting maximum flow velocity: 7.64 meters/second
sound speed in blood: 1540 m/s
frequency of ultrasound: 6 MHz
distance of the ultrasound transducer from the start of the measurement window: 25 mm
ultrasound oscillation cycles per sent-out ultrasonic PVD pulse: 10
resulting burst length (10 oscillations at 1540 m/s): 2.57 mm.

For an exemplary An ultrasonic pulse is sent out at the ultrasound element 19 and propagates in the direction of the measurement window 16. After sending out the pulse, the PWD system switches to the receiving direction and receives the portions that are continuously scattered back by scattering bodies in the blood, for example. The transit time of the pulse from the ultrasound element to the measurement window and from the measurement window back to the ultrasound element is taken into account in the process. In the case shown, the total relevant propagation path is thus 55.13 mm long (ultrasound element 19 to start of measurement window 16 plus burst length×2). The PWD system is switched back to transmission mode and the next pulse is sent out at the earliest when the last echo from the range of the measurement window 16 has arrived. In the specifically considered case, the pulse transit time limits the maximum pulse repetition rate to 27.93 kHz.

On the other hand, the maximum Doppler shift occurring in the case shown is 59.53 kHz. In a complex-value evaluation (IQ demodulation), this leads to a minimum pulse repetition rate of 59.53 kHz, in which the present Doppler shift can be interpreted without ambiguity. However, since the measurement is carried out with a maximum of 27.93 kHz (maximum pulse repetition rate; see above), the Nyquist sampling theorem is violated in this case and ambiguities generally occur in the resulting Doppler spectrum. In this case, these ambiguities are resolved using an operating parameter of the flow machine of the support system in order to be able to make a clear statement about the main flow velocity in the observation window.

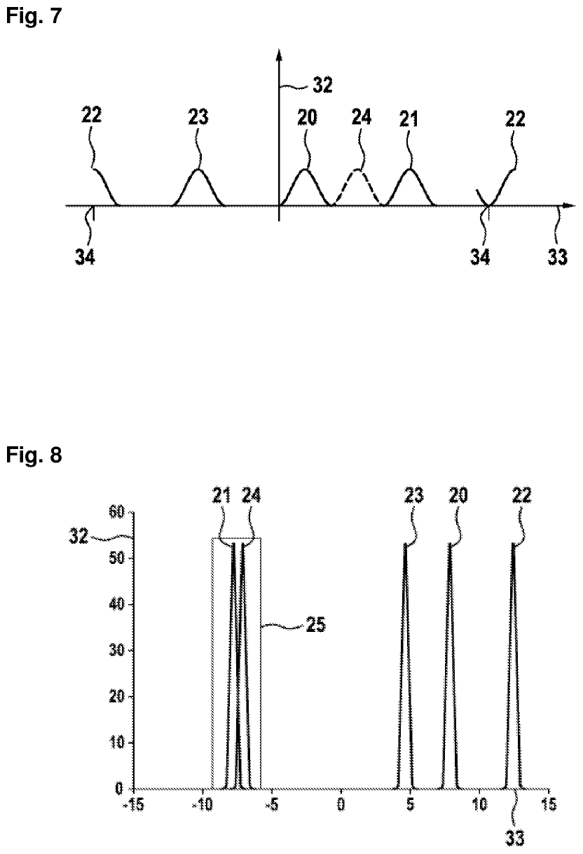

FIG. 7 schematically shows an exemplary Doppler frequency spectrum. Here, a schematic illustration of the previously presented relationships in the frequency spectrum is shown. A corresponding illustration of the shown relationships is also illustrated in FIG. 8.

FIG. 7 shows the amplitude 32 of the Doppler signal over the (averaged) frequency 33 with fixed pulse repetition rate 34 (PRF). The (fixed) pulse repetition rate 34 in the example considered here is 27 kHz. Simplified spectra for various flow velocities of the fluid (here: the blood) are shown in FIG. 7. A first flow velocity 20 is less than a second flow velocity 21, which in turn is less than a third flow velocity 22, which in turn is less than a fourth flow velocity 23, which in turn is less than a fifth flow velocity 24.

It can be seen that at the third flow velocity 22, there is already a violation of the Nyquist theorem, i.e., the Doppler frequency is in the range of the pulse repetition rate (PRF; here 27 kHz by way of example). With further increasing flow velocity of the blood, the spectrum moves from the negative frequency range to the coordinate origin. Here, there is already ambiguity about the direction of flow, i.e., either a fast flow toward the ultrasound element or a slower flow away from the ultrasound element. With further increasing flow velocity, the spectrum of the fifth flow velocity 24 appears in the ambiguity range of high or low flow velocity.

The solution presented here advantageously allows a resolution of such ambiguities. In principle, a comparatively rough range estimation can contribute to this purpose since the ultrasound method still works with high precision (resolution to 1-2 decimal places of the flow velocity in meters/second or of the volume flow in liters/minute), but ambiguity about the range of several meters/second or liters/minute is present.

FIG. 8 schematically shows another exemplary Doppler frequency spectrum.

FIG. 8 illustrates the problem and the resolution of ambiguity once again using the example with the parameters used above fᵤₛ=6 MHZ, PRF=27.93 kHz, C_{blood}=1540 m/s, and a windowing (window function) with a so-called Hamming window 25. The figure shows the frequency behavior at the following flow velocities:

$$first flow velocity 20 = - 1 m / s ,$$

$$second flow velocity 21 = + 1 m / s ,$$

$$third flow velocity 22 = 2 m / s ,$$

$$fourth flow velocity 23 = 3 m / s ,$$

$$fifth flow velocity 24 = 4.5 m / s .$$

In this context, a negative velocity means blood flowing toward the ultrasound element and appears in a frequency shift with a positive sign.

This example shows that the measured frequency peaks are very close to one another at flow velocities of 1 m/s and 4.5 m/s. This ambiguity can be resolved by the (a priori) knowledge of the approximate velocity on the basis of the operating parameter of the flow machine.

This approximate velocity interval Vint (plausible range of the flow velocity) can be resolved with the aid of the following formula to a corresponding Doppler shift or a Doppler shift interval f_{d,int}.

$${f}_{d , int} = \frac{2 \cdot {f}_{0}}{{c}_{Blut}} \cdot {v}_{int}$$

In the example, the corresponding Doppler shift interval is 31.95 kHz to 35.84 kHz. In order to shift the corresponding frequency interval into the frequency range that can be represented with the PRF used, the determined non-representable frequencies can be converted into the representable frequency range using the following formula (for positive flow velocities).

$${f}_{d , int , PRF} = ( {f}_{d , int} mod PRF ) - \frac{PRF}{2}$$

For the values shown in the example, the frequency interval that can be predicted by means of the operating parameter thus includes all frequencies between −9.95 kHz and −6.05 kHz. All frequencies measured in this interval correspond to velocities in the range of 4.1 m/s to 4.6 m/s.

The exact velocity (actual flow velocity) can be determined by a calculation from the number of spectral “wraps” with the aid of the operating parameter interval (frequency interval that can be predicted by means of the operating parameter) and successive back calculation from the measured frequency using the formulas already shown. A “wrap” here refers to the jump of a signal from the greatest positive representable frequency (f_{pRF}/2) to the representable negative frequency of highest magnitude (−f_{pRF}/2). The true frequency is determined according to the formula

$${f}_{d} = {nf}_{PRF} + {f}_{mess}$$

where the parameter n denotes the number of spectral “wraps.” For low flow velocities, f_{d}=fₘₑₐₛ; at higher velocities, ambiguities occur with regard to the value of n, which can be resolved according to the solution proposed here by additional knowledge (the operating parameter(s) of the flow machine).

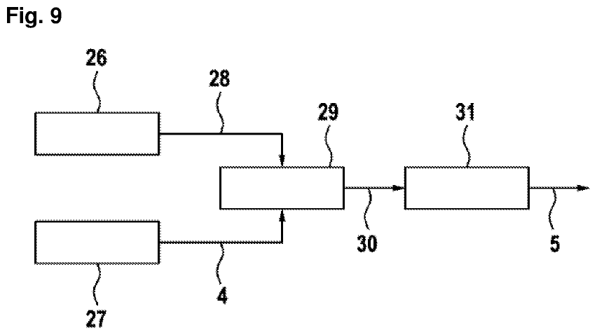

FIG. 9 schematically shows a functional illustration of a possible embodiment of the method presented here. The method in accordance with the illustration according to FIG. 9 serves to resolve the ambiguities. A PWD volume flow measurement 26 and a motor characteristic map-based volume flow measurement 27 take place in parallel or sequentially. The PWD volume flow measurement 26 can, for example, be carried out during step a). The motor characteristic map-based volume flow measurement 27 can be carried out, for example, between steps c) and d) or during step d). The PWD volume flow measurement 26 provides a Doppler spectrum 28. This can occur during step b), for example. The motor characteristic map-based volume flow measurement 27 provides an estimated (rough) fluid volume flow 4. This can occur, for example, between steps c) and d) or during step d). The Doppler spectrum 28 and the estimated fluid volume flow 4 are sent to an anti-aliasing unit 29. The anti-aliasing unit 29 determines from the estimated fluid volume flow 4 the (plausible) range in which the (actual) flow velocity is located, and from the Doppler spectrum 28 and the (plausible) volume flow range the corrected flow velocity 30, which is here also referred to as (actual) flow velocity through the support system. The anti-aliasing unit 29 can, for example, be a component of the processing unit also described here. A volume flow calculation unit 31 combines the known cross-sectional geometry and the known flow profiles determined in a construction type-specific and flow velocity-dependent manner for the (actual) fluid volume flow 5.

The PWD volume flow measurement 26 can comprise the following steps:
sending out an ultrasonic pulse,
waiting until the relevant echo of the measurement window,
receiving the echo of the measurement window,
possibly waiting for another waiting period until distant echoes die away,
sending out the next ultrasonic pulse.

The generated data can be stored temporarily in a memory for later evaluation or (e.g., with parallel implementation in programmable logic) can be further processed directly. While the ultrasonic pulse from the desired measurement window arrives (time limitation), the received echo sequence with the known ultrasonic pulse frequency is generally demodulated (“downmixing into the baseband”). Subsequently, the obtained baseband signal is generally transformed into the frequency range (transformation from time to frequency range for calculating the Doppler spectrum).

The motor characteristic map-based volume flow measurement 27 (rough volume flow measurement) can comprise the following steps:
determining a pump operating parameter, such as rotation rate (revolutions per minute, in short: RPM), power consumption, current consumption, and/or pressure difference across the flow machine (e.g., pump),
calculating the estimated fluid volume flow via a relationship determined in a type-specific manner or via interpolation (e.g., table-based interpolation) from a motor characteristic map determined in a type-specific manner.

The volume flow calculation unit 31, for example, carries out the following: multiplication of the known cross section in the range of the observation window 16 (formula symbol: A) with the flow velocity 30 (formula symbol: v), and a flow velocity-dependent flow profile correction parameter (formula symbol f(v)). In this case, the (actual) fluid volume flow (formula symbol Qₚ) can result according to the following formula:

$${Q}_{p} = f ( v ) \times v \times A$$

The anti-aliasing unit 29 and the volume flow calculation unit 31 can also be combined into one unit. In addition, the Doppler spectrum can be mapped directly to the volume flow Qₚ, for example.

The solution presented here allows in particular one or more of the following advantages:
highly accurate calculation of the pump volume flow by means of a Doppler ultrasonic sensor.
combination of a high-precision Doppler ultrasound measurement and a rough estimation on the basis of motor operating parameters (e.g., one or more of rotational speed, current, power, built-up pressure) allows the operation of the ultrasound measurement with violation of the Nyquist theorem (necessity arising from geometric conditions) and a subsequent resolution of arisen ambiguities with the aid of the rough estimate.

1. A method for determining a flow velocity of blood flowing through a cardiac support system, the method comprising:
carrying out a pulsed Doppler measurement of blood flowing through the cardiac support system using an ultrasonic sensor of the cardiac support system to determine a measurement result, the cardiac support system further comprising:
a flow machine; and
a processing unit configured to correct a possible ambiguity of the measurement result of the ultrasonic sensor based on at least one operating parameter of the flow machine to determine at least the blood flow velocity or the blood volume flow;
evaluating the measurement result to generate an evaluated measurement result, wherein the measurement result comprises the possible ambiguity;
determining the at least one operating parameter of the flow machine of the cardiac support system;
determining the blood flow velocity or the blood volume flow based on the evaluated measurement result; and
correcting the possible ambiguity of the measurement result using the at least one operating parameter.

2. The method of claim 1, wherein carrying out the pulse Doppler measurement comprises emitting a second ultrasonic pulse after an echo of a prior first ultrasonic pulse has died away.

3. The method of claim 1, wherein evaluating the measurement result comprises determining that the pulsed Doppler measurement has a pulse repetition rate of less than two times a maximum Doppler frequency shift of the blood flowing through the cardiac support system.

4. The method of claim 1, wherein the at least one operating parameter is based on at least one of the following: a rotational speed of a drive of the flow machine, an electrical current of the flow machine, an electrical power of the flow machine, and a differential pressure across the flow machine.

5. The method of claim 4, wherein determining the flow velocity is further based on the at least one operating parameter.

6. The method of claim 1, further comprising determining a plausible range in which plausible measurement results can be located based on the at least one operating parameter.

7. The method of claim 1, further comprising determining a fluid volume flow of the blood flowing through the cardiac support system based on the flow velocity.

8. The method of claim 1, wherein the at least one possible ambiguity comprises several possible flow velocities of the flowing blood.
